(19) United States
(12) Patent Application Publication
(10) Pub. No.: US 2015/0174194 A1
(43) Pub. Date: Jun. 25, 2015

(54) METHODS FOR TREATING LIVER TRANSPLANT RECIPIENTS

(71) Applicant: AbbVie Inc., North Chicago, IL (US)

(72) Inventors: Barry M. Bernstein, Mequon, WI (US); Eoin Coakley, Green Oaks, IL (US); Regis A. Vilchez, Lake Forest, IL (US)

(73) Assignee: ABBVIE INC., North Chicago, IL (US)

(21) Appl. No.: 14/574,654

(22) Filed: Dec. 18, 2014

Related U.S. Application Data
(60) Provisional application No. 61/918,264, filed on Dec. 19, 2013.
(60) Provisional application No. 61/972,866, filed on Mar. 31, 2014.
(60) Provisional application No. 62/008,784, filed on Jun. 6, 2014.

(51) Int. Cl. A61K 38/07; A61K 38/06; A61K 31/513; A61K 31/427

(52) U.S. Cl. CPC A61K 38/07; A61K 31/427; A61K 38/06; A61K 31/513

(57) ABSTRACT

This application features interferon-free therapies for the prevention or treatment of HCV infection in liver transplant recipient. In one aspect, the treatment comprises administering a DAA combination to a liver transplant recipient after liver transplantation, wherein the DAA combination comprises Compound 1, ritonavir, Compound 2 and Compound 4.

### FIELD OF THE TECHNOLOGY

This application relates to interferon-free treatment of liver transplant recipients for the prevention or treatment of hepatitis C virus (HCV) infection.

### BACKGROUND

The HCV is an RNA virus belonging to the Hepacivirus genus in the Flaviviridae family. The enveloped HCV virion contains a positive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

Chronic HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often incomplete.

### DETAILED DESCRIPTION

Recurrence of HCV infection after liver transplantation is universal and is a primary cause of graft loss. Current interferon-based HCV therapies have been associated with treatment-limiting toxicities and low efficacy. The application features the use of a combination of direct acting antiviral agents (DAAs), without the use of interferon, to treat liver transplant recipients for the prevention or treatment of HCV infection. The methods comprise administering a combination of DAAs to a liver transplant recipient.

In any aspect, embodiment, example, preference or feature described herein, the DAA combinations can be used, for example, with ribavirin. In any aspect, embodiment, example, preference or feature described herein, the DAA combinations can be used, for example, without ribavirin.

In any aspect, embodiment, example, preference or feature described herein, the treatment of liver transplant recipients can last from 4 to 20 weeks. For example, in any aspect, embodiment, example, preference or feature described herein, the treatment lasts for 4 weeks. For another example, in any aspect, embodiment, example, preference or feature described herein, the treatment lasts for 5 weeks. For another example, in any aspect, embodiment, example, preference or feature described herein, the treatment lasts for 6 weeks. For another example, in any aspect, embodiment, example, preference or feature described herein, the treatment lasts for 7 weeks. For another example, in any aspect, embodiment, example, preference or feature described herein, the treatment lasts for 8 weeks. For another example, in any aspect, embodiment, example, preference or feature described herein, the treatment lasts for 9 weeks. For another example, in any aspect, embodiment, example, preference or feature described herein, the treatment lasts for 10 weeks. For another example, in any aspect, embodiment, example, preference or feature described herein, the treatment lasts for 11 weeks. For another example, in any aspect, embodiment, example, preference or feature described herein, the treatment lasts for 12 weeks. For another example, in any aspect, embodiment, example, preference or feature described herein, the treatment lasts for 13 weeks. For another example, in any aspect, embodiment, example, preference or feature described herein, the treatment lasts for 14 weeks. For another example, in any aspect, embodiment, example, preference or feature described herein, the treatment lasts for 15 weeks. For another example, in any aspect, embodiment, example, preference or feature described herein, the treatment lasts for 16 weeks. For another example, in any aspect, embodiment, example, preference or feature described herein, the treatment lasts for 17 weeks. For another example, in any aspect, embodiment, example, preference or feature described herein, the treatment lasts for 18 weeks. For another example, in any aspect, embodiment, example, preference or feature described herein, the treatment lasts for 19 weeks. For another example, in any aspect, embodiment, example, preference or feature described herein, the treatment lasts for 20 weeks. For another example, in any aspect, embodiment, example, preference or feature described herein, the treatment lasts for 21 weeks. For another example, in any aspect, embodiment, example, preference or feature described herein, the treatment lasts for 22 weeks. For another example, in any aspect, embodiment, example, preference or feature described herein, the treatment lasts for 23 weeks. For another example, in any aspect, embodiment, example, preference or feature described herein, the treatment lasts for 24 weeks.

In any aspect, embodiment, example, preference or feature described herein, any DAA combination known in the art can be used to treat liver transplant recipients. Non-limiting examples of suitable DAA combinations can comprise one of the following combinations:
a combination of Compound 1, ritonavir, and Compound 4;
a combination of Compound 1, ritonavir, Compound 4 and Compound 2;
a combination of PSI-7977 and GS-5885;
a combination of PSI-7977 and TMC-435;
a combination of PSI-7977 and GS-5816;
a combination of PSI-7977 and BMS-790052;
a combination of BMS-790052 and BMS-650032;
a combination of GS-5885, GS-9190, and GS-9451; or
a combination of BI-201335 and BI-207127.

As used herein, ritonavir is not a DAA; instead, it is used to enhance the pharmacokinetics of Compound 1.

In any aspect, embodiment, example, preference or feature described herein, each DAA in the DAA combination can be, for example, dosed separately from the other DAA(s) in the DAA combination. In any aspect, embodiment, example, preference or feature described herein, each DAA in the DAA combination can be, for example, dosed together with the other DAA(s) in the DAA combination.

In any aspect, embodiment, example, preference or feature described herein, each DAA in the DAA combination can be, for example, formulated separately from the other DAA(s) in the DAA combination. In any aspect, embodiment, example, preference or feature described herein, all DAAs in the DAA combination can be, for example, formulated together in a single formulation.

In any aspect, embodiment, example, preference or feature described herein, one or more DAAs in the DAA combination can be, for example, dosed once daily (QD), and the other DAA(s) in the combination can be dosed twice daily (BID). In any aspect, embodiment, example, preference or feature described herein, all DAAs in the DAA combination can be, for example, dosed once daily.

In any aspect, embodiment, example, preference or feature described herein, a DAA can be in a pharmaceutically acceptable salt form.

Preferably, in any aspect, embodiment, example, preference or feature described herein, the DAA combination used to treat liver transplant recipients is a combination of Compound 1, ritonavir and Compound 4, all of which are formulated together in a single formulation and dosed once daily.

Also, preferably, in any aspect, embodiment, example, preference or feature described herein, the DAA combination used to treat liver transplant recipients is a combination of Compound 1, ritonavir and Compound 4, together with Compound 2, where Compound 1/ritonavir/Compound 4 are formulated together in a single formulation and dosed once daily, and Compound 2 is formulated separately and dosed twice daily.

In any aspect, embodiment, example, preference or feature described herein, Compound 1 can be used, for example, from 100-200 mg QD, preferably 150 mg QD.

In any aspect, embodiment, example, preference or feature described herein, Compound 2 can be used, for example, from 100 to 600 mg BID, preferably 250 mg BID.

In any aspect, embodiment, example, preference or feature described herein, Compound 4 can be used, for example, from 10 to 50 mg QD, preferably 25 mg BID.

In any aspect, embodiment, example, preference or feature described herein, ritonavir can be used, for example, from 50 to 200 mg QD, preferably 100 mg QD.

Compound 1

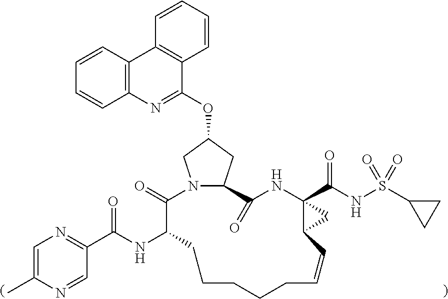 [SMILES: Cc1cnc(C(=O)N[C@H]2CCCCC/C=C\[C@@H]3C[C@@]3(C(=O)NS(=O)(=O)C3CC3)NC(=O)[C@@H]3C[C@@H](Oc4nc5ccccc5c5ccccc45)CN3C2=O)cn1]

is known as (2R,6S,13aS,14aR,16aS,Z)-N-(cyclopropylsulfonyl)-6-(5-methylpyrazine-2-carboxamido)-5,16-dioxo-2-(phenanthridin-6-yloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxamide. The synthesis and formulation of Compound 1 are described in U.S. Patent Application Publication Nos. 2010/0144608 and 2011/0312973 filed on Mar. 8, 2011, respectively.

Compound 2

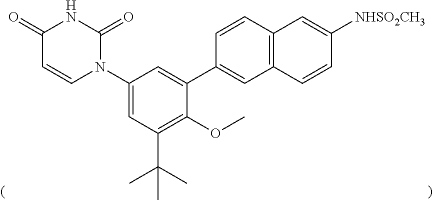

is known as N-(6-(3-tert-butyl-5-(2,4-dioxo-3,4-dihydropyrimidin-1(2H)-yl)-2-methoxyphenyOnaphthalen-2-yl)methanesulfonamide and is described in International Application Publication No. W02009/039127.

Compound 4

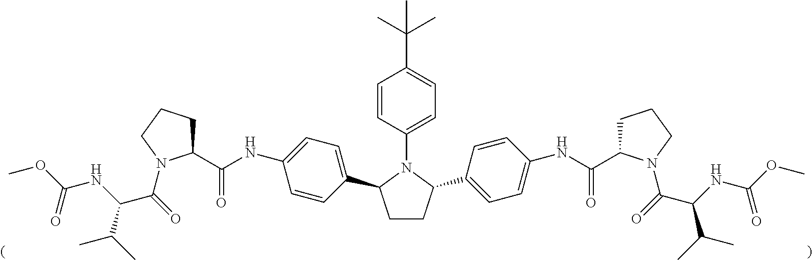

is known as dimethyl (2S,2′S)-1,1′-((2S,2′S)-2,2′-(4,4′-((2S,5S)-1-(4-tert-butylphenyl)pyrrolidine-2,5,diyl)bis(4,1-phenylene))bis(azanediyl)bis(oxomethylene)bis(pyrrolidine-2,1-diyObis(3 -methyl-1-oxobutane-2,1-diyl)dicarbamate, and is described in U.S. Publication No. 2010/0317568.

In any aspect, embodiment, example, preference or feature described herein, the DAA combination can be used with ribavirin (including a pro-drug thereof). Ribavirin may include any suitable form or formulation of ribavirin. Exemplary formulations of ribavirin include COPEGUS®, REBETOL® and RIBASPHERE®. An exemplary pro-drug of ribavirin is taribavirin having the chemical name of 1-β-D-ribofuranosyl-1,2,4-triazole-3-carboxamidine. Ribavirin and taribavirin may be administered in accordance with ribavirin and taribavirin administration well known in the art. In some embodiments, COPEGUS® or REBETOL® is administered in a daily dosage amount of from about 500 mg to about 1500 mg in one dose or in divided doses. In some embodiments, COPEGUS® or REBETOL® is administered in a daily dosage amount of about 800 mg. In some embodiments, REBETOL® is administered in a daily dosage amount of about 1000 mg. In some embodiments, COPEGUS® or REBETOL® is administered in a daily dosage amount of about 1200 mg. In some embodiments, REBETOL® is administered in a daily dosage amount of about 1400 mg. Suitable dosages of ribavirin are dependent on the weight of the subject, for example about 1000-1200 mg. Suitable total daily dosages of ribavirin include, but are not limited to about 400 mg to about 1400 mg a day, alternatively about 800 mg to about 1400 mg per day, alternatively about 400 mg to about 1200 mg, alternatively about 800 mg to about 1200 mg.

In any aspect, embodiment, example, preference or feature described herein, the DAA combination can be administered to liver transplant recipients before, during, or after liver transplantation for the prevention or treatment of HCV infection.

In any aspect, embodiment, example, preference or feature described herein, the HCV infection to be treated or prevented can be, for example, genotype 1 infection. The HCV infection can also be, for example, genotype 2 infection. The HCV infection can also be, for example, genotype 3 infection. The HCV infection can also be, for example, genotype 4 infection. The HCV infection can also be, for example, genotype 5 infection. The HCV infection can also be, for example, genotype 6 infection. The HCV infection can also be, for example, genotype la infection. The HCV infection can also be, for example, genotype 1b infection.

Preferably, in any aspect, embodiment, example, preference or feature described herein, the DAA combination is administered to liver transplant recipients after liver transplantation.

Preferably, in any aspect, embodiment, example, preference or feature described herein, the liver transplant recipient is infected with HCV genotype 1.

In any aspect, embodiment, example, preference or feature described herein, the liver transplant recipient can be cirrhotic or non-cirrhotic.

In any aspect, embodiment, example, preference or feature described herein, the liver transplant recipient can be, for example, either HCV-free or infected with HCV prior to the liver transplantation.

In any aspect, embodiment, example, preference or feature described herein, the liver transplantation can, for example, be a transplantation of the whole liver organ or a portion thereof.

Various measures may be used to express the effectiveness of the present methods of HCV treatment. One such measure is rapid virological response (RVR), meaning that HCV is undetectable in the subject after 4 weeks of treatment, for example, after 4 weeks of administration of two or more of DAAs and ribavirin. Another measure is the presence or absence of detectable virus at the end of therapy (EOT or EOTR). Another measure is (SVR), which, as used herein, means that the virus is undetectable at the end of therapy and for at least a number of weeks after the end of therapy (i.e., SVRx, where x is the number of weeks after the end of therapy).

It should be understood that the above-described embodiments and the following examples are given by way of illustration, not limitation. Various changes and modifications within the scope of the present application will become apparent to those skilled in the art from the present description.

### EXAMPLE 1

### Clinical Study

In an ongoing open-label phase 2 study, non-cirrhotic liver transplant recipients with recurrent HCV genotype 1 (GT1) infection received co-formulated Compound 1/ritonavir/Compound 4 (150 mg/100 mg/25 mg QD), together with Compound 2 (250 mg BID) and ribavirin, for 24 weeks. The patients were >12 months post-liver transplant, had not received treatment for HCV infection after transplantation, and had a screening biopsy confirmed Metavir score<F2. Because of potential interaction between the calcineurin inhibitor(CNI) and the therapy, the CNI dose was adjusted accordingly.

Thirty-four patients were enrolled in the study. Baseline characteristics/efficacy are in the table. Treatment-emergent adverse events were observed in 88.2% of patients and were generally mild with no severe events. One patient discontinued. Three patients received erythropoietin and none underwent transfusion. There were no episodes of rejection.

The result in Table 1 showed that the interferon-free regimen of Compound 1/ritonavir/Compound 4 plus Compound 2 and ribavirin, was generally well-tolerated and achieved high RVR, EOTR, and SVR4 rates in liver transplant recipients with recurrent HCV GT1 infection. High SVR12 rate is expected.

Further analysis showed that 96.8% of patients (30 out of the total 31 patients tested) achieved SVR4 and 95% (19 out of the total 20 patients tested) achieved SVR12.

TABLE 1
Baseline characteristics and efficacy.

| | Patients | |
|---|---|---|
| | N = 34 | |
| Time since transplantation - months, median (range) | 39.5 | (12.9-136.4) |
| Male, n (%) | 27 | (79.4) |
| Age - 3 yr, median (range) | 60.0 | (30.0-71.0) |
| Race, n (%) | | |
| Black race | 4 | (11.8) |
| White race | 29 | (85.3) |
| Hispanic or Latino ethnicity, n (%) | 6 | (17.6) |
| BMI - kg/m², median (range) | 30.1 | (19.3-36.9) |
| HCV subtype, n (%) | | |
| GT 1a | 29 | (85.3) |
| GT1b | 5 | (14.7) |
| Immunosuppressive medication, n (%) | | |
| Tacrolimus | 29 | (85.3) |
| Cyclosporine | 5 | (14.7) |
| IL28B CC, n (%) | 8 | (23.5) |
| Baseline HCV viral load - log₁₀ IU/mL, median | 6.7 | (5.3-7.6) |
| (range) | | |
| RVR, n/N (%) | 34/34 | (100) |
| EOTR, n/N (%) | 13/13 | (100) |
| SVR4, n/N (%) | 12/13 | (92.3) |

| | |
|---|---|
| Breakthrough, n | 0 |
| Relapse, n | 1 |

### EXAMPLE 2

### Dose Adjustment

Example 1 showed the safety and efficacy of 3D+RBV (i.e., co-formulated Compound 1/ritonavir/Compound 4 (150 mg/100 mg/25 mg QD), together with Compound 2 (250mg BID) and ribavirin) in liver transplant (LT) recipients with GT1 HCV infection.

The Example summarizes the management of concentrations of the immunosuppressants, tacrolimus (TAC) and cyclosporine (CSA) during the treatment. In healthy volunteers, the TAC and CSA concentrations, 24 hours after co-dosing with 3D, showed a 17- and 16-fold increase, respectively, necessitating dose adjustment.

Patients (n=34) on stable TAC or CSA therapy, at least 12 months post-LT, fibrosis stage <F2, received 3D +RBV for 24 weeks. When co-dosed with 3D, it was recommended that the prestudy total CSA daily dose be reduced to one-fifth and given QD. For TAC, a dose of 0.5 mg/7 days or 0.2 mg/3 days was recommended. Subsequent dose and dosing frequency modifications in TAC were made based on the individual TAC levels. Observed CSA trough concentrations (C_{trough}) were summarized for CSA recipients (n=5) while TAC data (n=29) were interrogated further to profile on-study TAC C_{trough}s. A nonlinear mixed effects model was used to characterize TAC concentration-time profiles using NONMEM software. A one compartment pharmacokinetic (PK) model with first order oral absorption and between subject variability on apparent clearance was used to describe the TAC data. The model estimated TAC PK parameters were used to predict TAC concentration-time profiles in order to evaluate TAC dosing strategies when co-dosed with 3D.

Blood concentrations of TAC (median: 4.6 ng/ml (interquartile range (IQR): 3.3-6.6 ng/ml, dose: 0.2-1 mg)) and CSA (median: 111 ng/ml (IQR: 92-138 ng/ml, dose: 25-75 mg)) were maintained when co-dosed with 3D. The median dosing interval was 10 days for TAC, and 1 day for CsA. Simulations demonstrated that subjects with a stable prestudy TAC C_{trough} of 6 ng/mL, while receiving the 3D with TAC 0.5 mg every 7 days or 14 days, would have TAC C_{trough} in the range of 6-9 ng/ml and 2-4 ng/ml, respectively. For LT recipients with GT1 HCV infection receiving 3D, the recommended CSA or TAC dose modifications yielded concentrations within the therapeutic range.

The present application also features the following:

What is clamed is

1. A method of treatment for preventing or treating HCV infection in a liver transplant recipient, comprising administering a combination of DAAs to said recipient, wherein said treatment does not comprise the use of interferon, and said treatment last from 4 to 24 weeks.

2. The method of claim 1, wherein said combination of DAAs comprises Compound 1, ritonavir, and Compound 4.

3. The method of claim 1, wherein said combination of DAAs comprises Compound 1, ritonavir, Compound 4 and Compound 2.

4. The method of claim 3, wherein the treatment lasts for 12 week.

5. The method of claim 3, wherein the treatment lasts for 24 week.

6. The method of claim 3, wherein the treatment comprises administering ribavirin to said recipient.

7. The method of claim 3, wherein the combination of DAAs is administered to said recipient after liver transplantation on said recipient.

8. The method of claim 3, wherein the recipient is HCV-free prior to receiving said liver transplant.

9. The method of claim 3, wherein the recipient is infected with HCV prior to receiving said liver transplant.
